(19) United States
(12) Patent Application Publication
(10) Pub. No.: US 2002/0155163 A1
(43) Pub. Date: Oct. 24, 2002

(54) INTEGRATED MULTI-VITAMIN AND MINERAL COMBINATION

(76) Inventors: SAMUEL D. BENJAMIN, EAST SETAUKET, NY (US); ANDREW WEIL, VAIL, AZ (US)

Correspondence Address: Richard B. Klar, 875 Avenue of the Americas, Suite 2301, NEW YORK, NY, 10001, US

(21) Appl. No.: 09/472,669

(22) Filed: Dec. 27, 1999

(51) Int. Cl. A61K 33/00; A61K 31/695; A61K 31/69; A61K 31/59; A61K 31/525; A61K 31/51; A61K 31/385; A61K 31/355; A61K 33/24; A61K 31/095

(52) U.S. Cl. 424/600; 424/617; 514/63; 514/64; 514/167; 514/168; 514/251; 514/276; 514/458; 514/440; 514/706

(57) ABSTRACT

A daily multi-vitamin and mineral combination for use in the adjunct care of humans with asthma comprising thiamin, riboflavin, niacin, Vitamin B₆, folate, Vitamin B₁₂, biotin, pantothenic acid, choline, inositol, para-amino benzoic acid, Vitamin C, calcium, magnesium, iodine, selenium, manganese, chromium, molybdenum, boron, zinc, potassium, silicon, sulfur, vanadium, citrus bioflavonoid complex, hesperidin complex, rutin, Vitamin A, Vitamin D, Vitamin E, lycopene, lutein, Coenzyme Q10 and N-acetyl cysteine. For adjunct care of humans with diabetes, alpha-lipoic acid is substituted for N-acetyl cysteine and the amount of inositol is increased. A method of supplementing the nutritional intake of humans with diabetes and/or asthma through daily oral administration of the appropriate multi-vitamin and mineral combination.

### FIELD OF THE INVENTION

The present invention relates to a daily integrated multi-vitamin and mineral combination, and more particularly pertains to a daily multi-vitamin and mineral combination containing phytonutrients that is useful in the adjunct care of asthma and/or diabetes.

### DISCUSSION OF THE RELATED ART

The use of multi-vitamin and mineral combination formulations to supplement the nutritional needs of humans is known. It is recognized that vitamins and minerals play important physiological roles, and that a deficiency or excess of certain vitamins and minerals has been linked to the etiology of certain diseases.

The B vitamins perform several well-known functions. Vitamin B₁, thiamin, helps maintain smooth muscle and helps in the formation of blood cells. It is necessary, also, for proper nervous system function. Vitamin B₂, riboflavin, is necessary for healthy hair, nails and mucous membranes. It also plays an important role in the formation of red blood cells and the production of antibodies. Vitamin B₃, niacin, helps in the production of most sex hormones, and also helps to lower cholesterol and maintain blood circulation. Vitamin B₆, pyridoxine, is involved in the synthesis of RNA and DNA, and helps relieve water retention in women. Folic acid is essential to the production of red blood cells and hormones, and is involved, as well, in DNA synthesis. Vitamin B₁₂, cyanocobalamin, is necessary for overall metabolism and nervous system function, and is essential for the metabolism of folic acid. It also is necessary to prevent anemia. Biotin is necessary for the metabolism of proteins, carbohydrates and fats, as well as for healthy hair and skin. Vitamin B₅, pantothenic acid, is important for the production of adrenal gland hormones. It is referred to as the “anti-stress vitamin.” Choline is necessary for nervous system and brain function, and is important in gall bladder and liver function. Inositol helps remove fats from arteries and from the liver, and has been noted to be necessary for brain function. Para-amino benzoic acid (“PABA”) aids in the metabolism of proteins and in the production of red blood cells.

Other vitamins exhibit other functions. Vitamin C has antioxidant properties. It is necessary for collagen synthesis, and has been shown to reduce the risk of heart disease, as well as reducing cervical epithelial abnormalities. Vitamin A prevents night blindness and other disorders, reduces the risk of lung cancer, and negates the effects of sun damage. It also possesses antioxidant properties. Vitamin D assists in the mineralization and calcification of bone, prevents osteomalacia in adults, and lowers the blood pressure. Vitamin E, along with exhibiting antioxidant properties, helps reduce the risk of lung cancer and heart disease.

Several minerals have also exhibited beneficial effects. Calcium builds bones and teeth, and is necessary for proper cardiac rhythms and for the conduction of nerve impulses. Iodine helps to metabolize fats, and is necessary for proper thyroid function. It also reduces fibrocystic breast conditions. Magnesium is involved in several enzymatic reactions in the body, and has been found beneficial in the treatment of cardiac arrhythmias and the management of diabetes. Selenium possesses antioxidant properties, and has been shown to reduce the risk of heart attack and heart disease. Manganese is involved in protein synthesis and central nervous system function, assists in bone growth, and helps prevent osteoporosis. It also regulates the production and release of insulin, assists in the metabolism of fats and proteins, and assists in the production of milk, as well as the thyroid hormone thyroxin. Chromium deficiency has been linked to weight loss and glucose intolerance, as well as to peripheral neuropathy. Chromium also helps lower LDL (low density lipoprotein) and raise HDL (high density lipoprotein) levels, and may protect against coronary artery disease. Molybdenum plays a biochemical role in enzyme function. It also helps metabolize fats, and plays a role in iron utilization. Boron is used to maintain healthy bones, and allows for increased absorption of calcium, magnesium and phosphorus. Potassium is necessary for proper fluid balance, as well as proper heartbeat and nerve transmission. Zinc is needed for proper formation of RNA and DNA, and also is necessary for growth and sexual development.

In addition to generally-known vitamins and minerals, other substances have been shown to exhibit beneficial physiological effects. Phytonutrients, such as bioflavonoids and carotenoids, are known to exhibit biological activity. Bioflavonoids reduce capillary fragility and protect biologically-important compounds through antioxidant activity. Bioflavonoids also exhibit anti-allergy properties. The bioflavonoid hesperidin is known to improve capillary permeability, and has been used in the treatment of neuropathies; while the bioflavonoid rutin is linked to strengthening of the capillaries and anti-inflammatory effects.

The carotenoid lycopene may reduce the risk of prostate and breast cancer. It has been inversely associated with the risk of cervical cancer in women. Also, the carotenoid lutein has been linked to a decrease in the risk of macular degeneration.

The use of multi-vitamin and mineral combinations to supplement the particular nutritional needs of persons with asthma, as an adjunct to other treatment for asthma, is known. It is recognized that the nutritional and health needs of persons with asthma differ in many respects from those of non-asthmatics. Asthma is a chronic inflammatory disorder of the airways leading to the lungs. During an asthma attack, the body's own immune cells, including eosinophils, mast cells, lymphocytes and macrophages, produce specific chemical substances called leukotrienes, that cause blood vessels to constrict and lung tissue to contract. In particular, leukotrienes are known to be potent spasmogens of the human trachea, bronchus and lung parenchymal strips. Leukotrienes also mediate mucous production. It is known that leukotrienes cause the biological reactions of asthma.

Certain substances are particularly beneficial with respect to the health of persons suffering from asthma. It is recognized that the mucolytic N-acetyl cysteine, which is the natural precursor of glutathione, acts as a natural inhibitor of certain leukotrienes. Decreased levels of glutathione have been linked to the occurrence of cataracts. The use of N-acetyl cysteine as a cytoprotective agent in cases of paracetamol toxicity also is well established.

The use of multi-vitamin and mineral combinations to supplement the particular nutritional needs of persons with diabetes, as an adjunct to other treatment for diabetes, is known. It is recognized that the nutritional and health needs of persons with diabetes differ in many respects from those of non-diabetics. Diabetes is characterized by an impaired ability to remove serum or plasma glucose after a glucose load, such as a meal. Long-term diabetes leads to complications including neuropathy (including numbness in the extremities), nephropathy (including renal failure), retinopathy and macrovasculopathy (including cardiac disease). Other effects of diabetes include blindness, birth defects and infant mortality.

Certain substances are particularly beneficial with respect to the health of persons suffering from asthma. It is recognized that diabetes and glucose intolerance can be treated with inositol triphosphate and/or D-chiro-inositol. Inositol has been shown to help diabetic neuropathy. Inositol also is a component of insulin-related inositol phosphoglycans, which have been shown to mediate insulin action. A deficiency of D-chiro-inositol has been linked to polycystic ovary syndrome, the most common cause of female infertility.

Alpha-lipoic acid, also known as thioct acid, has been shown to be effective in the treatment of diabetic and alcoholic polyneuropathy. This effect may be the result of an increased blood flow through nervous tissue, repairing damage. Alpha-lipoic acid also helps lower high-blood-sugar levels in diabetics by stimulating glucose utilization (and insulin sensitivity).

The prior art discloses multi-vitamin and mineral supplements containing many of the water- and fat-soluble vitamins, as well as many of the essential trace elements. However, the prior art does not disclose a multi-vitamin and mineral supplement for use in the adjunct care of humans with asthma, and in a form adapted to be administered orally (taken by mouth), via a minimal number of oral dosage forms, containing the following: the eight B-complex vitamins; the other B vitamins (choline, inositol, PABA); Vitamin C; the fat-soluble vitamins A, D and E; the trace elements calcium, iodine, magnesium, manganese, chromium, molybdenum, boron, zinc, potassium, silicon, sulfur and vanadium; the bioflavonoids hesperidin and rutin; the carotenoids lycopene and lutein; Coenzyme Q10; and N-acetyl cysteine.

What is needed is a high-potency daily multi-vitamin and mineral formulation for use in the adjunct care of humans with asthma that minimizes the number of dosages needed for optimal nutritional supplementation, yet contains nutritionally optimal amounts of each of the above-listed substances to be included in such a supplement.

Accordingly, the present invention provides a novel multi-vitamin and mineral combination, with phytonutrients, for use in the adjunct care of humans with asthma, and containing nutritionally optimal amounts of the eight B-complex vitamins; the other B vitamins (choline, inositol, PABA); Vitamin C; the fat-soluble vitamins A, D and E; the trace elements calcium, iodine, magnesium, manganese, chromium, molybdenum, boron, zinc, potassium, silicon, sulfur and vanadium; the bioflavonoids hesperidin and rutin; the carotenoids lycopene and lutein; Coenzyme Q10; and N-acetyl cysteine.

The prior art also does not disclose a multi-vitamin and mineral supplement for use in the adjunct care of humans with diabetes, and in a form adapted to be administered orally (taken by mouth), via a minimal number of oral dosage forms, containing the following: the eight B-complex vitamins; the other B vitamins (choline, inositol, PABA); Vitamin C; the fat-soluble vitamins A, D and E; the trace elements calcium, iodine, magnesium, manganese, chromium, molybdenum, boron, zinc, potassium, silicon, sulfur and vanadium; the bioflavonoids hesperidin and rutin; the carotenoids lycopene and lutein; Coenzyme Q10; and alpha-lipoic acid.

What is needed is a high-potency daily multi-vitamin and mineral formulation for use in the adjunct care of humans with diabetes that minimizes the number of dosages needed for optimal nutritional supplementation, yet contains nutritionally optimal amounts of each of the above-listed substances to be included in such a supplement.

Accordingly, the present invention provides a novel multi-vitamin and mineral combination, with phytonutrients, for use in the adjunct care of humans with diabetes, and containing nutritionally optimal amounts of the eight B-complex vitamins; the other B vitamins (choline, inositol, PABA); Vitamin C; the fat-soluble vitamins A, D and E; the trace elements calcium, iodine, magnesium, manganese, chromium, molybdenum, boron, zinc, potassium, silicon, sulfur and vanadium; the bioflavonoids hesperidin and rutin; the carotenoids lycopene and lutein; Coenzyme Q10; and alpha-lipoic acid.

The present invention also provides novel multi-vitamin and mineral combinations for use in the adjunct care of humans with asthma and/or diabetes, adapted to be taken orally, which minimize the number of oral dosage forms required to be ingested in order to receive the same nutritional benefit. The present invention also provides novel multi-vitamin and mineral combinations which reduce the risk of vitamin and mineral overdosing and toxicity.

### BRIEF SUMMARY OF THE INVENTION

The multi-vitamin and mineral combination of the present invention for use in the adjunct care of humans with asthma comprises vitamin B-complex vitamins, other B vitamins, Vitamin C, fat soluble vitamins, bioflavonoids, carotenoids, phytoestrogens, trace elements, Coenzyme Q10 and N-acetyl cysteine.

In a preferred embodiment, this multi-vitamin and mineral combination comprises approximately 50-100 mg thiamin, 45-55 mg riboflavin, 50-100 mg niacin, 50-100 mg Vitamin B₆, 400-800 μg folate, 80-120 μg Vitamin B₁₂, 80-220 μg biotin, 40-60 mg pantothenic acid, 40-60 mg choline, 40-60 mg inositol, 40-60 mg para-amino benzoic acid, 100-500 mg Vitamin C, 240-600 mg calcium, 120-275 mg magnesium, 120-180 μg iodine, 160-240 μg selenium, 0.8-1.2 mg manganese, 200-800 μg chromium, 60-90 μg molybdenum, 5-10 mg boron, 4.5-33 mg zinc, 0.8-1.2 mg potassium, 1.6-2.4 mg silicon, 4-6 mg sulfur, 8-12 μg vanadium, 32-48 mg citrus bioflavonoid complex, 3.2-4.8 mg hesperidin complex, 32-48 mg rutin, 15,000-25,000 IU Vitamin A, 320-480 IU Vitamin D, 400-880 IU Vitamin E, 4-6 mg lycopene, 4.8-7.2 mg lutein, 15-60 mg Coenzyme Q10, and 540-660 mg N-acetyl cysteine.

The present invention is also directed to a multi-vitamin and mineral combination for use in the adjunct care of humans with diabetes, which comprises vitamin B-complex vitamins, other B vitamins, Vitamin C, fat soluble vitamins, bioflavonoids, carotenoids, phytoestrogens, trace elements, Coenzyme Q10 and alpha-lipoic acid.

In a preferred embodiment, this multi-vitamin and mineral combination comprises approximately 50-100 mg thiamin, 45-55 mg riboflavin, 50-100 mg niacin, 50-100 mg Vitamin B₆, 400-800 μg folate, 80-120 μg Vitamin B₁₂, 80-220 μg biotin, 40-60 mg pantot 40-60 mg choline, 540-660 mg inositol, 40-60 mg para-amino benzoic acid, 100-500 mg Vitamin C, 240-600 mg calcium, 120-275 mg magnesium, 120-180 μg iodine, 160-240 μg selenium, 0.8-1.2 mg manganese, 200-800 μg chromium, 60-90 μg molybdenum, 5-10 mg boron, 4.5-33 mg zinc, 0.8-1.2 mg potassium, 1.6-2.4 mg silicon, 4-6 mg sulfur, 8-12 μg vanadium, 32-48 mg citrus bioflavonoid complex, 3.2-4.8 mg hesperidin complex, 32-48 mg rutin, 15,000-25,000 IU Vitamin A, 320-480 IU Vitamin D, 400-880 IU Vitamin E, 4-6 mg lycopene, 4.8-7.2 mg lutein, 15-60 mg Coenzyme Q10, and 90-110 mg alpha-lipoic acid.

The present invention is further directed at a method of supplementing the nutritional intake of humans with asthma and/or diabetes by the oral administration (taking by mouth) of the appropriate above-mentioned multi-vitamin and mineral combination. In a preferred embodiment, the components of each of the multi-vitamin and mineral combinations are divided into three formulations, each presented as an oral dosage form. The intended daily intake of both the multi-vitamin and mineral combination for use in the adjunct care of humans with asthma and the multi-vitamin and mineral combination for use in the adjunct care of humans with diabetes is one oral dosage form of each of the first, second and third formulations, taken together.

The oral dosage forms for all formulations may be a tablet or a softgel capsule. When in the form of a tablet, a formulation further comprises micro-crystalline cellulose, stearic acid and magnesium stearate. When in the form of a softgel capsule, a formulation further comprises soybean oil, gelatin, water, glycerin, beeswax and caramel.

### BRIEF DESCRIPTION OF THE DRAWINGS

Not Applicable.

### DETAILED DESCRIPTION OF THE INVENTION

Table I shows a formula of a multi-vitamin and mineral combination in accordance with the present invention.

TABLE I
Formula

| | Acceptable | | Acceptable | | Acceptable |
|---|---|---|---|---|---|
| Component | Range | Component | Range | Component | Range |
| Thiamin | 50-100 mg | Calcium | 240-600 mg | Vanadium | 8-12 μg |
| Riboflavin | 45-55 mg | Magnesium | 120-275 mg | Citrus bioflavo- | 32-48 mg |
| | | | | noid complex | |
| Niacin | 50-100 mg | Iodine | 120-180 μg | Hesperidin | 3.2-4.8 mg |
| | | | | complex | |
| Vitamin B₆ | 50-100 mg | Selenium | 160-240 μg | Rutin | 32-48 mg |
| Folate | 400-800 μg | Manganese | 0.8-1.2 mg | Vitamin A | 15,000-25,000 IU |
| Vitamin B₁₂ | 80-120 μg | Chromium | 200-800 μg | Vitamin D | 320-480 IU |
| Biotin | 80-220 μg | Molybdenum | 60-90 μg | Vitamin E | 400-880 IU |
| Pantothenic acid | 40-60 mg | Boron | 5-10 mg | Lycopene | 4-6 mg |
| Choline | 40-60 mg | Zinc | 4.5-33 mg | Lutein | 4.8-7.2 mg |
| Inositol | 40-60 mg | Potassium | 0.8-1.2 mg | Coenzyme Q10 | 15-60 mg |
| PABA | 40-60 mg | Silicon | 1.6-2.4 mg | — | — |
| Vitamin C | 100-500 mg | Sulfur | 4-6 mg | — | — |

The components are preferably be combined as follows: thiamin, riboflavin, niacin, Vitamin B₆, folate, Vitamin B₁₂, biotin, pantothenic acid, choline, inositol, para-amino benzoic acid, Vitamin C, calcium, magnesium, iodine, selenium, manganese, chromium, molybdenum, boron, zinc potassium, silicon, sulfur, vanadium, citrus bioflavonoid complex, hesperidin complex and rutin are combined in a first formulation; and Vitamin A, Vitamin D, Vitamin E, lycopene, lutein and Coenzyme Q10 are combined in a second formulation.

In a multi-vitamin and mineral combination preferably for use in the adjunct treatment of humans with asthma, an additional component is included: approximately 540-660 mg N-acetyl cysteine. This additional component takes the form of a third formulation.

In a multi-vitamin and mineral combination preferably for use in the adjunct treatment of humans with diabetes, the following additional components are combined in a third formulation:

approximately 90-110 mg alpha-lipoic acid and approximately 500-600 mg inositol. This inositol is in addition to the approximately 40-60 mg of inositol already combined in the first formulation.

Each formulation may take the oral dosage form of a tablet or capsule, such as a softgel capsule. When the oral dosage form is a tablet, the formulation also contains microcrystalline cellulose, stearic acid, and magnesium stearate. When the oral dosage form is a softgel capsule, the formulation also contains soybean oil, gelatin, water, glycerin, beeswax, and caramel. The daily dosage for both the multi-vitamin and mineral combination for use in the adjunct treatment of humans with asthma, as well as the multi-vitamin and mineral combination for use in the adjunct treatment of humans with diabetes, is one oral dosage form of the first formulation, one oral dosage form of the second formulation, and one oral dosage form of the third formulation, taken together.

The multi-vitamin and mineral combinations of the present invention may be formulated using any pharmaceutically acceptable form of the vitamins, trace elements and other nutrients discussed, supra. Preferred forms include: Vitamin C in the form of Ester C®, which employs natural Vitamin C metabolites to enhance absorption and retention; thiamin mononitrate; niacinamide; pyridoxine HCl; calcium pantothenate; iodine taken from kelp; yeast-bound selenium, so as not to interfere with Vitamin C absorption; calcium carbonate, which may be taken from rock, rather than the conventional oyster shell, to maintain the kosher nature of the compound; magnesium oxide; manganese, chromium, molybdenum and zinc as amino acid chelates; boron in the form of an amino acid complex; choline bitartrate; potassium sulfate; silicon dioxide; sulfur in the form of methylsulfonylmethane; vanadium as a chelate; bioflavonoids, including hesperidin complex and rutin, from citrus fruits; Vitamin A as mixed carotenoids from D. salina, where the mixed carotenoids may include alpha carotene, beta carotene, cryptoxanthin, zeaxanthin and lutein; Vitamin D from fish oil; Vitamin E from mixed natural tocopherols; lycopene incorporated from tomatoes; and extra lutein from marigold flowers. Specifically with regard to the multi-vitamin and mineral combination of the present invention for use in the adjunct care of humans with asthma, the preferred N-acetyl cysteine is super high quality.

In a preferred embodiment of the multi-vitamin and mineral combinations of the present invention, both for the adjunct care of humans with asthma and for the adjunct care of humans with diabetes, the first and third formulations preferably take the oral dosage form of a tablet, and the second formulation preferably takes the oral dosage form of a softgel capsule. The oil-soluble nutrients of the second general formulation are placed in a softgel capsule along with soybean oil, which maximizes absorption of lycopene and Coenzyme Q10.

Methods of rendering vitamin formulations into oral dosage forms are known and discussed in U.S. Pat. No. 5,494,678 (Paradissis et al.); U.S. Pat. No. 5,556,644 (Chandra); and U.S. Pat. No. 5,654,011 (Jackson et al. '011 patent); and are incorporated by reference herein. Specifically, in Paradissis et al., the nutritional components are normally blended with conventional excipients, such as binders; lubricants, such as stearic acid; diluents, such as mannose; disintegrants, such as carboxymethyl cellulose; suspending agents, such as polyvinyl alcohol; absorbents, such as silicon dioxide; preservatives, such as sodium benzoate; surfactants, such as polysorbate 80; and colorants, such as F.D. & C. dyes. (U.S. Pat. No. 5,494,678, Col. 9, 11. 47-59). In Chandra, the pure nutrient substances are dry blended using a powder mill unit until they are mixed to form a resultant multinutrient powder. The blending is effected under conditions yielding a particle size of less than 50 microns. A sufficient amount of the resulting powder is then packed into opaque nonallergenic capsule shells. (U.S. Pat. No. 5,556,644, Col. 5, 11. 24-29). The dietary supplements of Jackson et al. may be formulated using any pharmaceutically acceptable form of the vitamins, minerals and other ingredients of the invention, including their salts. They may be formulated into capsules, tablets, powders, gels or liquids. (U.S. Pat. No. 5,654,011, Col. 7, 11. 50-67).

From these references and the examples below, various preparation processes should be apparent to one of ordinary skill in the art. For example, a manufacturer may formulate the tablet generally as follows: All ingredients are in dry form, and blending takes place in a V-Mixer. Proper distribution of the ingredients is assured by use of conventional trituration. Stearic acid and magnesium stearate are added to the vitamins and minerals as binding agents. The mixed ingredients are measured and weighed, using a conventional tablet press and incorporating a volumetric process. Tablets are periodically weighed throughout the process to assure proper tablet fill. Finished tablets are sprayed with a water solution of microcrystalline cellulose as a coating. The tablets are then dried in a rotating drum.

As a further example, a manufacturer may formulate the softgel capsule as follows: Vitamins D and/or E are in oil form. All other ingredients are in dry form. Blending takes place in a heated vessel, with the ingredients agitated by impellers inside the vessel. Soybean oil is added for fluid consistency; beeswax is added as a suspending agent. Shell ingredients (gelatin, water, glycerin and caramel) are blended in a heated vessel. Fill ingredients and shell ingredients are kept heated together throughout the encapsulating process. The proper quantity and/or volume of blended ingredients is measured by conventional encapsulating equipment. Capsules are weighed periodically throughout the encapsulation process to assure proper fill. Finished capsules are dried for several days in a temperature and humidity controlled environment.

While the invention has been described with respect to certain specific embodiments, it will be appreciated that many modifications and changes may be made by those skilled in the art without departing from the invention. It is intended, therefore, by the appended claims to cover all such modifications and changes as may fall within the true spirit and scope of the invention.

What is claimed is:

1. A multi-vitamin and mineral combination for use in the adjunct care of humans with asthma comprising:
a. approximately 50-100 mg thiamin;
b. approximately 45-55 mg riboflavin;
c. approximately 50-100 mg niacin;
d. approximately 50-100 mg Vitamin B₆;
e. approximately 400-800 μg folate;
f. approximately 80-120 μg Vitamin B₁₂;
g. approximately 80-220 μg biotin;
h. approximately 40-60 mg pantothenic acid;
i. approximately 40-60 mg choline;
j. approximately 40-60 mg inositol;
k. approximately 40-60 mg para-amino benzoic acid;
l. approximately 100-500 mg Vitamin C;
m. approximately 240-600 mg calcium;
n. approximately 120-275 mg magnesium;
o. approximately 120-180 μg iodine;
p. approximately 160-240 μg selenium;
q. approximately 0.8-1.2 mg manganese;
r. approximately 200-800 μg chromium;
s. approximately 60-90 μg molybdenum;
t. approximately 5-10 mg boron;
u. approximately 4.5-33 mg zinc;
v. approximately 0.8-1.2 mg potassium;
w. approximately 1.6-2.4 mg silicon;
x. approximately 4-6 mg sulfur;
y. approximately 8-12 μg vanadium;
z. approximately 32-48 mg citrus bioflavonoid complex;
aa. approximately 3.2-4.8 mg hesperidin complex;
bb. approximately 32-48 mg rutin;
cc. approximately 15,000-25,000 IU Vitamin A;
dd. approximately 320-480 IU Vitamin D;
ee. approximately 400-880 IU Vitamin E;
ff. approximately 4-6 mg lycopene;
gg. approximately 4.8-7.2 mg lutein;
hh. approximately 15-60 mg Coenzyme Q10; and
ii. approximately 540-660 mg N-acetyl cysteine.

2. The multi-vitamin and mineral combination of claim 1, wherein said thiamin, riboflavin, niacin, Vitamin B₆, folate, Vitamin B₁₂, biotin, pantothenic acid, choline, inositol, para-amino benzoic acid, Vitamin C, calcium, magnesium, iodine, selenium, manganese, chromium, molybdenum, boron, zinc potassium, silicon, sulfur, vanadium, citrus bioflavonoid complex, hesperidin complex, and rutin are combined in a first formulation; said Vitamin A, Vitamin D, Vitamin E, lycopene, lutein and Coenzyme Q10 are combined in a second formulation; and said N-acetyl cysteine is in a third formulation.

3. A method of supplementing nutritional intake of humans with asthma by administering the multi-vitamin and mineral combination of claim 2.

4. The method of claim 3, wherein said first formulation of said multi-vitamin and mineral composition is administered orally.

5. The method of claim 4, wherein said first formulation of said multi-vitamin and mineral composition is administered in the form of a tablet and further comprises micro-crystalline cellulose, stearic acid and magnesium stearate.

6. The method of claim 3, wherein said second formulation of said multi-vitamin and mineral composition is administered orally.

7. The method of claim 6, wherein said second formulation of said multi-vitamin and mineral combination is administered in the form of a softgel capsule and further comprises soybean oil, gelatin, water, glycerin, beeswax, and caramel.

8. The method of claim 3, wherein said third formulation of said multi-vitamin and mineral composition is administered orally.

9. The method of claim 8, wherein said third formulation of said multi-vitamin and mineral composition is administered in the form of a tablet and further comprises micro-crystalline cellulose, stearic acid and magnesium stearate.

10. A multi-vitamin and mineral combination for use in the adjunct care of humans with diabetes comprising:
a. approximately 50-100 mg thiamin;
b. approximately 45-55 mg riboflavin;
c. approximately 50-100 mg niacin;
d. approximately 50-100 mg Vitamin B₆;
e. approximately 400-800 μg folate;
f. approximately 80-120 μg Vitamin B₁₂;
g. approximately 80-220 μg biotin;
h. approximately 40-60 mg pantothenic acid;
i. approximately 40-60 mg choline;
j. approximately 540-660 mg inositol;
k. approximately 40-60 mg para-amino benzoic acid;
l. approximately 100-500 mg Vitamin C;
m. approximately 240-600 mg calcium;
n. approximately 120-275 mg magnesium;
o. approximately 120-180 μg iodine;
p. approximately 160-240 μg selenium;
q. approximately 0.8-1.2 mg manganese;
r. approximately 200-800 μg chromium;
s. approximately 60-90 μg molybdenum;
t. approximately 5-10 mg boron;
u. approximately 4.5-33 mg zinc;
v. approximately 0.8-1.2 mg potassium;
w. approximately 1.6-2.4 mg silicon;
x. approximately 4-6 mg sulfur;
y. approximately 8-12 μg vanadium;
z. approximately 32-48 mg citrus bioflavonoid complex;
aa. approximately 3.2-4.8 mg hesperidin complex;
bb. approximately 32-48 mg rutin;
cc. approximately 15,000-25,000 IU Vitamin A;
dd. approximately 320-480 IU Vitamin D;
ee. approximately 400-880 IU Vitamin E;
ff. approximately 4-6 mg lycopene;
gg. approximately 4.8-7.2 mg lutein;
hh. approximately 15-60 mg Coenzyme Q10; and
ii. approximately 90-100 mg alpha-lipoic acid.

11. The multi-vitamin and mineral combination of claim 10, wherein said thiamin, riboflavin, niacin, Vitamin B₆, folate, Vitamin B₁₂, biotin, pantothenic acid, choline, para-amino benzoic acid, Vitamin C, calcium, magnesium, iodine, selenium, manganese, chromium, molybdenum, boron, zinc potassium, silicon, sulfur, vanadium, citrus bioflavonoid complex, hesperidin complex, rutin, and approximately 40-60 mg of said inositol are combined in a first formulation; said Vitamin A, Vitamin D, Vitamin E, lycopene, lutein and Coenzyme Q10 are combined in a second formulation; and said alpha-lipoic acid and approximately 500-600 mg of said inositol are combined in a third formulation.

12. A method of supplementing nutritional intake of humans with diabetes by administering the multi-vitamin and mineral combination of claim 11.

13. The method of claim 12, wherein said first formulation of said multi-vitamin and mineral composition is administered orally.

14. The method of claim 13, wherein said first formulation of said multi-vitamin and mineral composition is administered in the form of a tablet and further comprises micro-crystalline cellulose, stearic acid and magnesium stearate.

15. The method of claim 12, wherein said second formulation of said multi-vitamin and mineral composition is administered orally.

16. The method of claim 15, wherein said second formulation of said multi-vitamin and mineral combination is administered in the form of a softgel capsule and further comprises soybean oil, gelatin, water, glycerin, beeswax, and caramel.

17. The method of claim 12, wherein said third formulation of said multi-vitamin and mineral composition is administered orally.

18. The method of claim 17, wherein said third formulation of said multi-vitamin and mineral composition is administered in the form of a tablet and further comprises micro-crystalline cellulose, stearic acid and magnesium stearate.

19. The multi-vitamin and mineral combination of claim 1 or claim 10, wherein said Vitamin C is incorporated as Ester C®.

20. The multi-vitamin and mineral combination of claim 1 or claim 10, wherein said selenium is yeast-bound.

21. The multi-vitamin and mineral combination of claim 1 or claim 10 wherein said sulfur is incorporated as methylsulfonylmethane.
